(19) United States
(12) Patent Application Publication
(10) Pub. No.: US 2008/0255125 A1
(43) Pub. Date: Oct. 16, 2008

(54) Topically Applied Medicament for Animals

(75) Inventors: Dirk Mertin, Langenfeld (DE); Gerald Beddies, Dusseldorf (DE); Iris Heep, Koln (DE); Nikolaus Kowollik, Merzenich-Girbelsrath (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 10/585,608

(22) PCT Filed: Jan. 7, 2005
(86) PCT No.: PCT/EP2005/000067

§ 371 Date: Aug. 10, 2007

(30) Foreign Application Priority Data
Jan. 10, 2004 (DE) 102004001558.9

(51) Int. Cl. A61K 31/53; A61K 31/44; A61K 31/496; A61K 31/437

(52) U.S. Cl. 514/241; 514/353; 514/253.08; 514/300

(57) ABSTRACT

The invention relates to pharmaceutical preparations, which are applied to the pelt or skin of animals and are then absorbed by the latter orally.

The invention relates to pharmaceutical preparations which are applied to the coat or the skin of animals and which the latter then take up orally.

In animals, the oral administration of pharmaceuticals depends on the taste properties of the active compound and on the formulation. In the case of domestic animals, the administration of bitter-tasting active compounds, such as fluoroquinolones and praziquantel, is particularly difficult. On the other hand, there is a great need for palatable oral medicinal forms which the domestic animal takes up voluntarily from the hand of the animal's owner or from a feed bowl. As a rule, the animal's owner administers oral pharmaceuticals in one of the following ways: in the case of what is termed the poke-down method, the pharmaceutical is laid on the base of tongue and the mouth is then closed. The head is moved into the normal position and the throat is gently massaged until the medicinal form is swallowed. Occasionally, small quantities of liquid are also administered in order to facilitate the swallowing. In the second method, the medicinal form is hidden in a portion of feed and then administered. This method is unsuitable if the active compound has to be administered in the fasting state or the highly bitter inherent taste of the active compound overlays the taste of the feed. More rarely, the medicinal form is comminuted and strewn over the feed or dissolved in water.

Whereas these modes of use are frequently successful in dogs, which usually swallow immediately after oral uptake, cats are far more difficult to treat. Since they retain the medicinal form, or the feed which is provided with it, in the mouth for a relatively long time, a formulation constituent having an unpleasant taste has adequate opportunity to come into contact with the oral mucosa. The unpleasant taste then frequently leads to the pharmaceutical, or at least parts of it, being expectorated immediately. In order to facilitate the administration of semisolid preparations (pastes) in cats, it is sometimes recommended that these preparations should be applied to the paw, from where they can be licked off. However, this type of use is very unreliable since the pastes frequently do not adhere well to the coat and can be shaken off. Attempts to improve the palatability by adding a flavour are likewise rarely successful in cats since the unpleasant taste cannot be completely masked.

It has now been found, surprisingly, that an active compound-containing preparation, which is preferably of liquid consistency and which gives rise to severe defensive reactions after having been administered perorally into the oral cavity of a cat, is taken up voluntarily, and virtually completely, when it is applied to the coat of the animal. Evidently, the grooming reflex, which is controlled by the central nervous system, is so pronounced in cats that even the repulsive taste of the active compound is unable to prevent the active compound being taken up by the grooming. It can even be assumed that the grooming reflex is stimulated precisely by constituents of the pharmaceutical which have a bad taste, with the reflex only abating when the active compound has been completely removed from the coat and has consequently been taken up orally.

The invention therefore relates:

to a pharmaceutical preparation for use in animals, which is applied to the coat or the skin of the animal and which the latter then takes up orally.

The invention furthermore relates:

to a method for applying pharmaceutical active compounds in animals, in which a pharmaceutical preparation comprising the corresponding active compound is applied topically to the animal and the animal then takes up orally the applied pharmaceutical preparation.

In principle, any preparations which can be applied topically and which are also acceptable for an oral administration come into consideration as preparations which are suitable in accordance with the invention. Those which may be mentioned are: liquid, semi-liquid or pasty, and also solid, preparations. Liquid preparations are particularly preferred.

The topical application takes place, for example, in the form of dipping, spraying, bathing, washing, pouring-on, spotting-on and rubbing-in.

Solutions, emulsions and suspensions are suitable preparations.

Solutions for topical application are dripped on, painted on, rubbed in, sprayed on, sprinkled on or applied by immersion (dipping, bathing or washing).

The preparations according to the invention are preferably applied topically to the trunk, in particular, for example, to the back or to the flanks of the animal.

Solutions are prepared by dissolving the active compound in a suitable solvent and adding any possible additives such as solubilizers, acids, bases, buffer salts, antioxidants or preservatives.

Solvents which may be mentioned are: water, alkanols, glycols, polyethylene glycols, polypropylene glycols, glycerol, aromatic alcohols such as benzyl alcohol, phenylethanol, phenoxyethanol, esters such as ethyl acetate, butyl acetate and benzyl benzoate, ethers such as alkylene glycol alkyl ether, dipropylene glycol monomethyl ether and diethylene glycol monobutyl ether, ketones such as acetone and methyl ethyl ketone, aromatic and/or aliphatic hydrocarbons, vegetable or synthetic oils, such as medium-chain triglycerides or propylene glycol esters with medium-chain fatty acids, DMF, dimethylacetamide, N-methylpyrrolidone and 2-dimethyl-4-oxymethylene-1,3-dioxolane, as well as mixtures of the aforementioned solvents. Vegetable or synthetic oils, and their mixtures with the said solvents, are particularly suitable.

Solubilizers which may be mentioned are: solvents which promote the solution of the active compound in the main solvent or prevent its precipitation. Examples are polyvinylpyrrolidone, polyoxyethylated castor oil and polyoxyethylated sorbitan esters.

Examples of preservatives are benzyl alcohol, n-butanol, trichlorobutanol, p-hydroxybenzoic esters, benzoic acid, propionic acid and sorbic acid.

The solutions can be used directly. Concentrates are used after having been previously diluted down to the concentration for use.

It may be advantageous to add thickeners during the preparation. Thickeners are: inorganic thickeners such as bentonites, colloidal salicic acid and aluminium monostearate, and organic thickeners such as cellulose derivatives, xanthan, carrageenan, alginates, starch, gelatin, polyvinyl alcohols and their copolymers, acrylates and methacrylates.

Dyes are any dyes which are authorized for use on animals and which can be dissolved or suspended.

Antioxidants are sulphites or metabisulphites, such as sodium sulphite and potassium metabisulphite, ascorbic acid, butylhydroxytoluene, butylhydroxyanisol and tocopherol.

Photostabilizers are, for example, substances belonging to the benzophenone or novantisolic acid class.

Tackifiers are, for example, cellulose derivatives, xanthan, carrageenan, alginates, starch, gelatin, polyvinyl alcohols and their copolymers, acrylates and methacrylates.

Emulsions are either of the water-in-oil type or of the oil-in-water type.

They are prepared by dissolving the active compound either in the hydrophobic phase or in the hydrophilic phase and homogenizing this latter with the solvent of the other phase using suitable emulsifiers and, where appropriate, additional auxiliary substances such as dyes, preservatives, antioxidants, photostabilizers and viscosity-increasing substances.

Hydrophobic phases (oils) which may be mentioned are: paraffin oils, silicone oils, natural vegetable oils such as sesame oil, almond oil or castor oil, synthetic triglycerides such as caprylic/capric acid diglyceride, a triglyceride mixture containing vegetable fatty acid having a chain length of C₈₋₁₂, or other specially selected natural fatty acids, partial glyceride mixtures of saturated and unsaturated, and possibly also hydroxyl group-containing, fatty acids, and mono- and diglycerides of the C₈/C₁₀ fatty acids.

Fatty acid esters such as ethyl stearate, di-n-butyryl adipate, hexyl laurate, and dipropylene glycol pelargonate, esters of a branched fatty acid of medium chain length with saturated fatty alcohols having a chain length of C₁₆-C₁₈, isopropyl myristate, isopropyl palmitate, caprylic/capric acid esters of saturated fatty alcohols having a chain length of C₁₂-C₁₈, isopropyl stearate, oleyl oleate, decyl oleate, ethyl oleate, ethyl lactate, waxy fatty acid esters such as artificial duck uropygial gland fat, dibutyl phthalate, diisopropyl adipate, ester mixtures related to the latter, etc.

Fatty alcohols such as isotridecyl alcohol, 2-octyldodecanol, cetylstearyl alcohol and oleyl alcohol.

Fatty acids such as oleic acid and its mixtures.

Hydrophilic phases which may be mentioned are:

water, alcohols such as propylene glycol, glycerol and sorbitol and their mixtures.

Emulsifiers which may be mentioned are: nonionic surfactants, e.g. polyoxyethylated castor oil, polyoxyethylated sorbitan monooleate, sorbitan monostearate, glycerol monostearate, polyoxyethyl stearate and alkylphenol polyglycol ethers;

ampholytic surfactants such as di-Na-N-lauryl-β-iminodipropionate or lecithin;

anionic surfactants such as Na-lauryl sulphate, fatty alcohol ether sulphates, and the monoethanolamine salt of mono/dialkyl polyglycol ether orthophosphoric esters;

cationic surfactants such as cetyltrimethylammonium chloride.

Other auxiliaries which may be mentioned are: substances which increase viscosity and stabilize the emulsion, such as carboxymethyl cellulose, methyl cellulose and other cellulose and starch derivatives, polyacrylates, alginates, gelatin, gum arabic, polyvinylpyrrolidone, polyvinyl alcohol, copolymers composed of methyl vinyl ether and maleic anhydride, polyethylene glycols, waxes and colloidal salicic acid, or mixtures of the listed substances.

Suspensions are prepared by suspending the active compound in a carrier liquid, where appropriate in the added presence of other auxiliaries such as wetting agents, dyes, preservatives, antioxidants and photostabilizers.

Carrier liquids which may be mentioned are any homogeneous solvents and solvent mixtures.

Wetting agents (dispersing agents) which may be mentioned are the above-specified surfactants.

Other auxiliaries which may be mentioned are those which are specified above.

The preparations according to the invention have to fulfil all the conditions for a topical pharmaceutical preparation and also be suitable for oral uptake.

In order to ensure good oral uptake, the preparation which is applied to the coat should adhere to it. A particular consistency, as exhibited, for example, by the examples according to the invention, is desirable for this purpose. The viscosity of the preparations according to the invention is therefore preferably from 1 to 1000 mPa*s, particularly preferably from 10 to 500 mPa*s. If the viscosity is too low, there is a risk of the formulation dripping off the coat. On the other hand, highly viscous formulations can only be applied with difficulty. In addition to this, highly viscous preparations frequently only adhere to the coat inadequately and fall off, or are shaken off, before they can be taken up by the animal.

It is furthermore desirable for the preparation to have good spreadability so that it can also be used on a site on the coat which is difficult for the grooming to access. Good spreading furthermore leads to the preparation being distributed over a larger area of the coat. In this case, the animal requires more time to take up orally the quantity of active compound which has been applied, resulting in the inflow into the body being retarded and the dwell time, and thus the activity time, being prolonged. Kinetic investigations have demonstrated this therapeutically desirable prolongation of the dwell time in the body (see FIG. 1 and FIG. 2). The examples according to the invention exhibit good spreadability.

According to the invention, particular preference is given to what are termed spot-on formulations, in which small volumes, usually less than 10 ml, preferably 5 ml or less, of pharmaceutical are applied topically to the animal. The composition then spreads on the surface of the animal.

Usually, only a relatively small oral uptake is to be expected when only small volumes are applied since the grooming reflex would be more likely to be stimulated by high quantities of preparation, which the animal then regards as being dirt. Surprisingly, high levels of active compound in the blood were obtained even after applying only very small volumes. Thus, only about 1 ml of formulation was applied in Examples 2 and 3. Nevertheless, the plasma levels are comparable with those obtained with Example 1, which was applied in a volume of 4 ml (see FIG. 2). The preparations according to the invention consequently permit high oral availability even when only low volumes are applied.

The pharmaceutical is intended to be administered by the veterinarian in accordance with the instructions or else intended for subsequent administration by the animal's owner at home. A strongly smelling or staining preparation would be upsetting for the animal's owner. A repulsive odour, or any discoloration of the coat or skin and/or environment should therefore be avoided in the case of the preparations according to the invention.

It is consequently also possible to deliver pharmaceuticals having a bad taste in a simple and reliable manner using the mode of application according to the invention.

The preparations according to the invention are preferably employed in the case of animals which have a grooming reflex or a grooming behaviour which favours uptake. While the preparations are used, in particular in mammals, e.g. cats, dogs, rabbits, hares, guinea pigs, hamsters, mice and rats, they are also used in birds. Particular preference is given to using them in rabbits and, in particular, cats.

In principle, any active compounds which are suitable for topical application and oral uptake come into consideration as active compounds for the preparations according to the invention.

The following may be mentioned by way of example:

quinolone and related antibiotics, as are disclosed, inter alia, in the following documents: U.S. Pat. No. 4,670,444 (Bayer AG), U.S. Pat. No. 4,472,405 (Riker Labs), U.S. Pat. No. 4,730,000 (Abbott), U.S. Pat. No. 4,861,779 (Pfizer), U.S. Pat. No. 4,382,892 (Daiichi), U.S. Pat. No. 4,704,459 (Toyama), of which the following specific examples may be mentioned: benofloxacin, binfloxacin, cinoxacin, ciprofloxacin, danofloxacin, difloxacin, enoxacin, enrofloxacin, fleroxacin, gatifloxacin, ibafloxacin, levofloxacin, lomefloxacin, marbofloxacin, moxifloxacin, norfloxacin, ofloxacin, orbifloxacin, pefloxacin, pipemidic acid, pradofloxacin, temafloxacin, tosufloxacin, sarafloxacin, sparfloxacin.

Penicillins, cephalosporins and related β-lactams, such as amoxicillin, ampicillin, azidocillin, aztreonam, benzylpenicillin, cefaclor, cefadroxil, cefalexin, cefetamet pivoxil, cefixime, cefodizime, cefotiam, cefpodox improxetil, cefsulodin, ceftibuten, ceftizoxime, cefuroxime, clavulanic acid, dicloxacillin, flucloxacillin, imipenem, loracarbef, mezlocillin, oxacillin, phenoxymethylpenicillin, propicillin, sultamicillin, tazobactam.

Preference is likewise given to using the analgesics aceclofenac, acemetacin, acetylsalicylic acid, buprenorphine, carprofen, celecoxib, codeine, deracoxib, diclofenac, dihydrocodeine, felbinac, fentanyl, flufenamic acid, flunixin, flupirtine, flurbiprofen, hydromorphone, ibuprofen, indomethacin, ketoprofen, lonazolac, meclofenamic acid, mefenamic acid, meloxicam, metamizol, methadon, mofebutazone, morphine, naproxen, nefopam, niflumic acid, oxaprozin, oxycodon, paracetamol, parecoxib, pentazocin, pethidine, phenazone, phenylbutazone, piroxicam, piritramide, proglumetacin, propyphenazone, rofecoxib, tepoxalin, tiaprofenic acid, tilidin, tolfenamic acid, tramadol, valdecoxib, vedaprofen.

It is furthermore possible to use the following active compounds 4-aminosalicylic acid, abacavir, abamectin, acamprosate, acebutolol, acepromazine, acetylcysteine, aciclovir, acitretin, adapalene, albendazole, alendronic acid, alfuzosin, alprostadil, aluminium chloride, aluminium oxide, amantadine, ambroxol, amidotrizoic acid, amlodipine, amorolfine, amphotericin B, ascorbic acid, atenolol, atorvastatin, azithromycin, baclofen, benazepril, betamethasone, bezafibrate, bifonazole, biotin, bisoprolol, brivudine, bromhexine, bumetanide, bupranolol, calcium acetate, calcium carbonate, candesartan, captopril, carbidopa, carbocisteine, carteolol, carvedilol, celiprolol, cerivastatin, cetirizine, chenodeoxycholic acid, quinine, chlorambucil, chloramphenicol, chlormadinone, chloroquine, chlortalidone, chlortetracycline, ciclosporin, cidofovir, cilastatin, cilazapril, clarithromycin, clenbuterol, clindamycin, clodronic acid, clomipramine, dapsone, dexamethasone, didanosine, diethylcarbamazine, dipotassium clorazepate, diltiazem, dinoprost, diphenhydramine, doramectin, doxazosin, doxycycline, dutasteride, econazole, efavirenz, emodepside, enalapril, ephedrine, eprinomectin, eprosartan, erythromycin, esmolol, etacrynic acid, ethambutol, etidronic acid, famciclovir, fenbendazole, fendiline, fenticonazole, fexofenadine, finasteride, florfenicol, flubendazole, fluconazole, flucytosine, flumethasone, fluvastatin, folic acid, fosfestrol, fosfomycin, fosinopril, fumaric acid, furosemide, gabapentin, gallopamil, ganciclovir, gemfibrozil, halofantrine, heparin, hyaluronic acid, hydrochlorothiazide, hydrocortisone hydrogen succinate, ibandronic acid, iloprost, imidapril, indinavir, irbesartan, isoconazole, isoniazide, itraconazole, ivermectin, josamycin, potassium canrenoate, kanamycin, ketoconazole, ketotifen, lamivudine, leflunomide, levocabastine, levodopa, levothyroxine, linezolid, lincomycin, lipoic acid, lisinopril, lodoxamide, loperamide, lopinavir, losartan, mebendazole, medroxyprogesterone, mefloquine, megestrol, melarsoprol, mepindolol, mesalazine, mesna, metamizole, metergoline, methionine, methotrexate, methylprednisolone, metoclopramide, metoprolol, metronidazole, miconazole, minocycline, moexipril, montelukast, moxidectin, nadolol, sodium dibunate, naftifine, Na picosulphate, natamycin, nateglinide, nelfinavir, neomycin, nevirapin, nicardipine, nicergoline, niclosamide, nicotinic acid, nifedipine, nifuratel, nifirpirinol, nifurtimox, nimodipine, nimorazole, nisoldipine, nitrofurantoin, nitroxoline, nystatin, olsalazine, omeprazole, orotic acid, oseltamivir, oxamniquine, oxfendazole, oxibendazole, oxiconazole, oxprenolol, oxybutynin, oxytetracycline, pamidronic acid, pangamic acid, penbutolol, penicillamine, pentamidine, perindopril, phenobarbital, phenoxybenzamine, phenylpropanolamine, pimobendan, piretanide, ponazuril, pravastatin, praziquantel, prednisolone, primaquine, probenecide, progesterone, proglumide, proguanil, proligestone, propentofylline, propiverine, propanolol, pyrantel embonate, pyrazinamide, pyrimethamine, pyrvinium embonate, quinapril, ramipril, repaglinide, reviparin, ribarvirin, rifabutin, rifampicin, risedronic acid, roxithromycin, saquinavir, selamectin, selegilin, sevelamer, sotalol, spectinomycin, spiramycin, spirapril, stavudine, streptomycin, sulfachlorpyridazine, sulfadiazine, sulfadimethoxine, sulfadimidine, sulfadoxine, sulfalene, sulfamethoxazole, sulfanilamide, sulfasalazine, talinolol, tamsulosin, teicoplanin, telithromycin, telmisartan, tenofovir disoproxil, terazosin, terbinafin, tetracycline, tetroxoprim, theophylline, thiabendazole, tiagabine, tiludronic acid, tinidazole, tioconazole, tolterodine, toltrazuril, trandolapril, tranexamic acid, tretinoin, triamcinolone acetonide, triclabendazole, trimethoprim, tripelenamine, tromantadine, trospium chloride, tryptophan, ursodeoxycholic acid, valaciclovir, valproic acid, vancomycin, verapamil, vidarabin, vigabatrin, zalcitabine, zidovudine, and zoledronic acid.

The abovementioned compounds can also be used in the form of their esters or salts. Hydrates of the compounds are also included in accordance with the invention. Pharmaceutically utilizable salts are to be understood, for example, as being the salts of hydrochloric acid, sulphuric acid, acetic acid, glycolic acid, lactic acid, succinic acid, citric acid, tartaric acid, maleic acid, methanesulphonic acid, 4-toluenesulphonic acid, galacturonic acid, gluconic acid, embonic acid, glutamic acid or aspartic acid. Furthermore, compounds can also be bound to acidic or basic ion exchangers. Pharmaceutically utilizable basic salts which may be mentioned are the alkali metal salts, for example the sodium or potassium salts, the alkaline earth metal salts, for example the magnesium or calcium salts, the zinc salts, the silver salts and the guanidinium salts.

Hydrates are understood as being both the hydrates of the free compounds themselves and the hydrates of their salts.

The active compounds can also be present in the preparations admixed with synergists or in combination with other active compounds.

### EXAMPLES

### Example 1

1.5 g of flupirtine base are dissolved in a mixture composed of 40 g of propylene glycol dicaprylate/dicaprate (Miglyol 840) and 40 g of isopropanol. 3.5 g of the same mixture are used to make up to 100 ml. This results in a clear solution having a flupirtine concentration of 1.5% m/v.

In each case 4 ml were applied to several sites on the backs of 4 healthy cats (15-20 mg of flupirtine base/kg of bodyweight (BW)). Blood samples were withdrawn after 0, 0.5, 1, 2, 4, 6, 10, 24, 30 and 48 hours and analysed by HPLC.

The following plasma concentrations were obtained:

TABLE 1
Plasma levels of flupirtine following application of 4 ml of the
formulation in accordance with Example 1 to the backs of cats, n = 4,
dose, 15-20 mg of flupirtine base/kg of BW

| Time after | Plasma concentration of flupirtine base [μg/L] | | | | |
|---|---|---|---|---|---|
| application [h] | Cat 8 | Cat 81 | Cat 20 | Cat 16 | Mean value |
| 0 | <LoQ | <LoQ | <LoQ | <LoQ | <LoQ |
| 0.5 | 84 | 33 | 163 | 976 | 314 |
| 1 | 402 | 171 | 249 | 1982 | 701 |
| 2 | 2536 | 3430 | 2535 | 3563 | 3016 |
| 4 | 4191 | 4530 | 7170 | 3471 | 4840 |
| 6 | 3164 | 2688 | 5615 | 2244 | 3428 |
| 10 | 2858 | 1734 | 4039 | 1977 | 2652 |
| 24 | 1969 | 583 | 3223 | 910 | 1671 |
| 30 | 1531 | 384 | 2157 | 574 | 1161 |
| 48 | 582 | 76 | 818 | 188 | 416 |

<LoQ = below the determination limit (10 μg/L)

### Example 2

0.2 g of sodium sulphite is dissolved in 8 g of water; 90 g of propylene glycol are added and 3 g of flupirtine maleate are suspended in the mixture. After the mixture has been adjusted to pH 6 with 2.35 g of 2 N sodium hydroxide solution, the active compound dissolves completely. The final volume is made up to 100 ml with 1.15 g of water. This results in a clear solution having a flupirtine maleate concentration of 3.0% m/v.

In each case one volume, corresponding to a flupirtine maleate dose of 10 mg/kg of bodyweight, was applied to a site on the backs of 4 healthy cats. Blood samples were withdrawn after 0, 0.5, 1, 2, 3, 4, 6, 10, 24, 30 and 48 hours and analysed by HPLC. The following plasma concentrations were obtained:

TABLE 2
Plasma levels of flupirtine following application of a formulation
corresponding to Example 2 to the backs of cats, n = 4, dose, 10 mg
of flupirtine maleate/kg of BW

| Time after | Plasma concentration of flupirtine base [μg/L] | | | | |
|---|---|---|---|---|---|
| application [h] | 2911C | 2903C | 2930C | 2923C | Mean value |
| 0 | <LoQ | <LoQ | <LoQ | <LoQ | <LoQ |
| 0.5 | 46 | 24 | 22 | 119 | 53 |
| 1 | 2963 | 48 | 125 | 561 | 924 |
| 2 | 2429 | 69 | 155 | 1501 | 1039 |
| 3 | 3002 | 1100 | 421 | 1829 | 1588 |
| 4 | 2515 | 801 | 356 | 1642 | 1329 |
| 6 | 1199 | 330 | 154 | 946 | 657 |
| 10 | 814 | 556 | 117 | 661 | 537 |
| 24 | 386 | 323 | 28 | 284 | 255 |
| 30 | 191 | 130 | 11 | 162 | 124 |
| 48 | 128 | 22 | <LoQ | 87 | 61 |

<LoQ = below the determination limit (10 μg/L)

### Example 3

3.0 g of flupirtine maleate are suspended in 92.2 g of medium-chain triglycerides (Miglyol 812) and dispersed using a rotor-stator homogenizer (Ultra-Turrax). This results in 100 ml of a suspension having a flupirtine maleate concentration of 3.0% m/v.

In each case one volume, corresponding to a flupirtine maleate dose of 10 mg/kg of bodyweight, was applied to a site on the backs of 4 healthy cats. Blood samples were withdrawn after 0, 0.5, 1, 2, 3, 4, 6, 10, 24, 30 and 48 hours and analysed by HPLC. The following plasma concentrations were obtained:

TABLE 3
Plasma levels of flupirtine following application of a formulation
corresponding to Example 3 on the backs of cats, n = 4, dose, 10 mg
of flupirtine maleate/kg of BW

| Time after | Plasma concentration of flupirtine base [μg/L] | | | | |
|---|---|---|---|---|---|
| application [h] | 2911C | 2903C | 2930C | 2923C | Mean value |
| 0 | <LoQ | <LoQ | <LoQ | <LoQ | <LoQ |
| 0.5 | 274 | 383 | 74 | 140 | 218 |
| 1 | 640 | 614 | 780 | 307 | 585 |
| 2 | 1464 | 1232 | 869 | 739 | 1076 |
| 3 | 2012 | 1707 | 529 | 1239 | 1372 |
| 4 | 2536 | 1952 | 931 | 1911 | 1833 |
| 6 | 3400 | 2375 | 949 | 2404 | 2282 |
| 10 | 4658 | 1701 | 1037 | 1615 | 2253 |
| 24 | 2112 | 573 | 663 | 1148 | 1124 |
| 30 | 2184 | 371 | 289 | 429 | 818 |
| 48 | <LoQ | <LoQ | <LoQ | <LoQ | <LoQ |

<LoQ = below the determination limit (10 μg/L)

The same formulation was applied to the same animals at the same dose one day after a castration operation. Blood samples were withdrawn after 0, 0.5, 1, 2, 4, 6, 10, 24, 30 and 48 hours and analysed by HPLC. The following plasma concentrations were obtained:

TABLE 4
Plasma levels of flupirtine following application of a formulation
corresponding to Example 3 on the backs of cats after a sterilization
operation, n = 4, dose, 10 mg of flupirtine maleate/kg of BW

| Time after | Plasma concentration of flupirtine base [μg/L ] | | | | |
|---|---|---|---|---|---|
| application [h] | 2911C | 2903C | 2930C | 2923C | Mean value |
| 0 | <LoQ | <LoQ | <LoQ | <LoQ | <LoQ |
| 0.5 | <LoQ | 12 | <LoQ | <LoQ | <LoQ |
| 1 | <LoQ | 22 | <LoQ | <LoQ | <LoQ |
| 2 | <LoQ | 16 | <LoQ | <LoQ | <LoQ |
| 4 | 60 | 30 | 23 | 45 | 39 |
| 6 | 273 | 32 | 51 | 246 | 151 |
| 10 | 331 | 258 | 143 | 265 | 249 |
| 24 | 1106 | 1067 | 338 | 780 | 835 |
| 30 | 673 | 680 | 261 | 417 | 508 |
| 48 | 356 | 333 | 299 | 200 | 297 |

<LoQ = below the determination limit (10 μg/L)

FIG. 1 summarizes the plasma levels obtained following application of the examples according to the invention and compares them with the plasma level obtained following the peroral administration of a tablet (dose, 4 mg of flupirtine maleate/kg of BW). The pharmacokinetic data are more readily compared by normalizing the different doses to a standard dose of 1 mg of flupirtine base/kg of BW (FIG. 2). Plasma active compound concentrations which corresponded to those obtained after peroral administration of a tablet were found in the case of all the examples according to the invention. The delayed grooming behaviour which is seen after an operation in this case shifts the tₘₐₓ markedly from 3-6 hours to 24 hours. The maximum concentrations are also lower due to the delayed uptake. In order to ensure post operative analgesia, the application should take place at a period of time before the operation which is sufficient for the animal to still be able to take up therapeutically relevant quantities.

The data show that, after an active compound-containing formulation has been applied to a cat's coat, it is almost completely taken up orally due to the grooming behaviour. In this way, even active compounds having a bad taste, such as flupirtine, fluoroquinolones or praziquantel, can be administered perorally in a reliable manner.

### FIGURES

FIG. 1: Plasma concentration of flupirtine following application of active compound-containing preparations to the coats of cats (n=4-8)

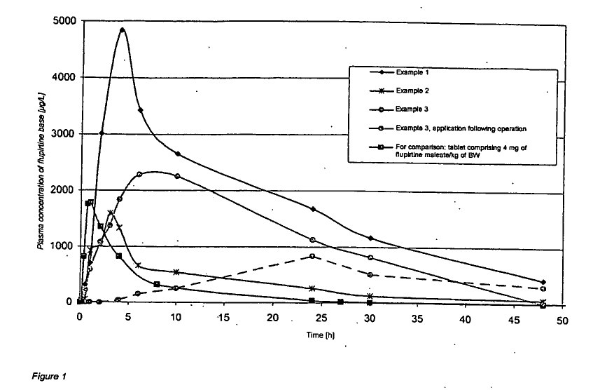

FIG. 2: Plasma concentration of flupirtine following application of active compound-containing preparations to the coats of cats (n=4-8), data normalized to a dose of 1 mg of flupirtine base/kg of BW

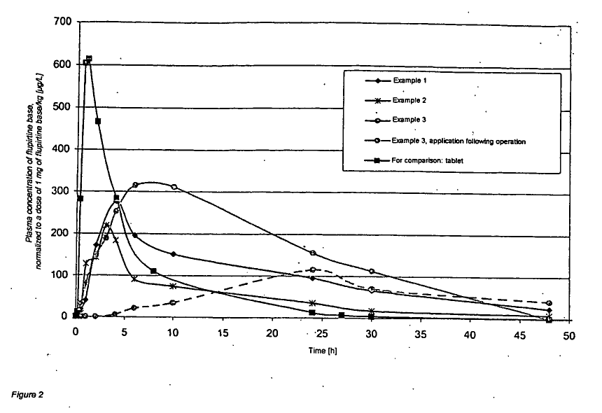

### Example 4

3.75 g of ponazuril are suspended in 44.25 g of glycerol and dispersed using a rotor-stator homogenizer. This results in 50 ml of a suspension having a ponazuril concentration of 7.5% m/m.

### Example 5

0.75 g of pradofloxacin is suspended in 49.25 g of polyethylene glycol 400 and dispersed using a rotor-stator homogenizer. This results in 50 ml of a suspension having a pradofloxacin concentration of 1.5% m/m.

### Example 6

1.25 g of enrofloxacin are suspended in 48.75 g of medium-chain triglycerides (Miglyol 812) and dispersed using a rotor-stator homogenizer. This results in 50 ml of a suspension having an enrofloxacin concentration of 2.5% m/m.

A volume corresponding to an enrofloxacin dose of approx. 5 mg/kg of bodyweight was applied to a site in the region of the backline between the shoulder blades of each of 4 healthy cats. At the listed times, blood samples were withdrawn and serum aliquots were analysed by HPLC. Until 4 hours after application, the animals wore a neck collar which was intended to prevent any licking/grooming of the application site. The following serum concentrations of enrofloxacin and the active metabolite ciprofloxacin were obtained:

TABLE 5
Serum concentrations of enrofloxacin following application of
0.7-0.9 ml of the formulation corresponding to Example 6 on the
backs of cats, n = 4, dose, approx. 5 mg of enrofloxacin/kg of BW,
neck collars removed at 4 hours after application

| | |
|---|---|
| Serum concentration of | |
| enrofloxacin [μg/L] | |
| in animal No.: | Mean |

| Time | 0463 | 0464 | 0510 | 0504 | [μg/L] |
|---|---|---|---|---|---|
| Prior to | <LoQ | <LoQ | <LoQ | <LoQ | <LoQ |
| appl. | | | | | |
| 1 h | <LoQ | <LoQ | <LoQ | <LoQ | <LoQ |
| 2 h | <LoQ | <LoQ | <LoQ | <LoQ | <LoQ |
| 4 h | <LoQ | <LoQ | <LoQ | <LoQ | <LoQ |
| 5 h | 135 | 97 | 504 | 706 | 361 |
| 6 h | 94 | 85 | 664 | 733 | 394 |
| 8 h | 86 | 66 | 483 | 516 | 288 |
| 10 h | 80 | 68 | 433 | 419 | 250 |
| 14 h | 126 | — | 303 | 327 | 252 |
| 28 h | 36 | 30 | 63 | 90 | 55 |
| 34 h | <LoQ | <LoQ | 28 | 40 | <LoQ |
| 52 h | <LoQ | <LoQ | <LoQ | <LoQ | <LoQ |

TABLE 6
Serum concentrations of ciprofloxacin following application of
0.7-0.9 ml of the formulation corresponding to Example 6 on the
backs of cats, n = 4, dose, approx. 5 mg of enrofloxacin/kg of BW,
neck collars removed at 4 hours after application

Serum concentration of
ciprofloxacin [μg/L]
in animal No.:

| Time | 0463 | 0464 | 0510 | 0504 | Mean [μg/L] |
|---|---|---|---|---|---|
| Prior to | <LoQ | <LoQ | <LoQ | <LoQ | <LoQ |
| appl. | | | | | |
| 1 h | <LoQ | <LoQ | <LoQ | <LoQ | <LoQ |
| 2 h | <LoQ | <LoQ | <LoQ | <LoQ | <LoQ |
| 4 h | <LoQ | <LoQ | <LoQ | <LoQ | <LoQ |
| 5 h | <LoQ | <LoQ | 57 | 64 | 37 |
| 6 h | <LoQ | <LoQ | 79 | 81 | 46 |
| 8 h | <LoQ | <LoQ | 71 | 70 | 42 |
| 10 h | <LoQ | <LoQ | 83 | 70 | 45 |
| 14 h | <LoQ | — | 82 | 73 | 56 |
| 28 h | <LoQ | <LoQ | 28 | 34 | <LoQ |
| 34 h | <LoQ | <LoQ | <LoQ | <LoQ | <LoQ |
| 52 h | <LoQ | <LoQ | <LoQ | <LoQ | <LoQ |

The data show that, after an active compound-containing formulation has been applied to the coats of cats, the substance is taken up orally as a result of the grooming behaviour; no percutaneous uptake was seen.

### Example 7

7.5 g of toltrazuril are suspended in 92.5 g of paraffin subliquidum and dispersed using a rotor-stator homogenizer. This results in 100 ml of a suspension having a toltrazuril concentration of 7.5% m/m.

### Example 8

4.0 g of toltrazuril are suspended in 96 g of sesame oil and dispersed using a rotor-stator homogenizer. This results in 100 ml of a suspension having a toltrazuril concentration of 4% m/m.

A volume corresponding to a toltrazuril dose of approx. 15 mg/kg of bodyweight was applied to a site in the region of the backline between the shoulder blades of each of 4 healthy cats. At the listed times, blood samples were withdrawn and serum aliquots were analysed by HPLC. Until 4 hours after application, the animals wore a neck collar which was intended to prevent any licking of the application site. The following serum concentrations of toltrazuril and the active metabolite toltrazuril sulphone were obtained:

TABLE 7
Serum concentrations of toltrazuril following application of
0.6-0.7 ml of the formulation corresponding to Example 8 on the
backs of cats, n = 4, dose, approx. 15 mg of toltrazuril/kg of BW; neck
collars removed at 4 hours after application

| | Serum concentration of | | | | |
|---|---|---|---|---|---|
| | toltrazuril [μg/L] in animal No.: | | | | |
| Time | 0472 D | 0470 D | 0493 D | 0494 D | Mean [μg/L] |
| Prior to | <LoQ | <LoQ | <LoQ | <LoQ | <LoQ |
| appl. | | | | | |
| 1 h | 42 | <LoQ | <LoQ | <LoQ | <LoQ |
| 2 h | 142 | <LoQ | <LoQ | <LoQ | 45 |
| 4 h | 214 | 188 | <LoQ | <LoQ | 107 |
| 5 h | 417 | 397 | 319 | 358 | 373 |
| 6 h | * | 617 | 1006 | 1063 | 895 |
| 8 h | 383 | 539 | 1134 | 1579 | 909 |
| 10 h | 539 | 617 | 1171 | 1590 | 979 |
| 14 h | 684 | 918 | 1074 | 1623 | 1075 |
| 28 h | 2035 | 1204 | 1763 | 5335 | 2584 |
| 34 h | 1442 | 898 | 1369 | 3980 | 1922 |
| 52 h | 1717 | 893 | 1906 | 2853 | 1842 |

TABLE 8
Serum concentrations of toltrazuril sulphone following application of
0.6-0.7 ml of the formulation corresponding to Example 8 on the
backs of cats, n = 4, dose, approx. 15 mg of toltrazuril/kg of BW; neck
collars removed at 4 hours after application

| | Serum concentration of toltrazuril | | | | |
|---|---|---|---|---|---|
| | sulphone [μg/L] in | | | | |
| | animal No.: | | | | |
| Time | 0472 D | 0470 D | 0493 D | 0494 D | Mean [μg/L] |
| Prior to | <LoQ | <LoQ | <LoQ | <LoQ | <LoQ |
| appl. | | | | | |
| 1 h | <LoQ | <LoQ | <LoQ | <LoQ | <LoQ |
| 2 h | <LoQ | <LoQ | <LoQ | <LoQ | <LoQ |
| 4 h | <LoQ | <LoQ | <LoQ | <LoQ | <LoQ |
| 5 h | <LoQ | <LoQ | <LoQ | <LoQ | <LoQ |
| 6 h | <LoQ | <LoQ | <LoQ | <LoQ | <LoQ |
| 8 h | <LoQ | <LoQ | <LoQ | <LoQ | <LoQ |
| 10 h | <LoQ | <LoQ | <LOQ | <LoQ | <LoQ |
| 14 h | <LoQ | <LoQ | 60 | 43 | 32 |
| 28 h | 130 | 122 | 308 | 397 | 239 |
| 34 h | 164 | 143 | 418 | 597 | 331 |
| 52 h | 470 | 389 | 809 | 1521 | 797 |

The data show that, after application of an active compound-containing formulation to the coats of cats, the substance is taken up orally as a result of the grooming behaviour; no percutaneous uptake was seen. Serum levels which were measured before the collar was removed very probably result from a minor oral uptake due to licking of the neck collar's inner side which has come into contact with the application site.

One volume each, corresponding to a toltrazuril dose of 8 mg/kg of bodyweight, was applied to a site in the flank region of 4 healthy rabbits. At the listed times, blood samples were withdrawn and serum aliquots were analysed by HPLC. Until 4 hours after application, the animals were fixed in a restraining device which prevented any licking of the application site. The following serum concentrations of toltrazuril and the active metabolite toltrazuril sulphone were obtained:

TABLE 9
Serum concentrations of toltrazuril following application of 1 ml of
the formulation corresponding to Example 8 to the flanks of rabbits,
n = 4, dose, 10.7-11.2 mg of toltrazuril/kg of BW; the animals were
fixed until 4 hours after application

| | |
|---|---|
| Serum concentration of | |
| toltrazuril [μg/L] | |
| in animal No.: | Mean |

| Time | 2564 | 2589 | 2548 | 2562 | [μg/L] |
|---|---|---|---|---|---|
| Prior to | <LoQ | <LoQ | <LoQ | <LoQ | <LoQ |
| appl. | | | | | |
| 1 h | <LoQ | <LoQ | 103 | <LoQ | 35 |
| 2 h | <LoQ | <LoQ | 104 | <LoQ | 35 |
| 4 h | <LoQ | <LoQ | 100 | <LoQ | 34 |
| 5 h | <LoQ | <LoQ | 452 | 25 | 126 |
| 6 h | 56 | 47 | 856 | 91 | 263 |
| 8 h | 232 | 245 | 2143 | 949 | 892 |
| 10 h | 484 | 897 | 3018 | 1486 | 1471 |
| 14 h | 1329 | 1409 | 3735 | 1717 | 2048 |
| 28 h | 2992 | 2548 | 4692 | 3035 | 3317 |
| 34 h | 4112 | 2955 | 4420 | 2767 | 3564 |
| 52 h | 3843 | 3014 | 4245 | 4308 | 3853 |

TABLE 10
Serum concentrations of toltrazuril sulphone following application of
1 ml of the formulation corresponding to Example 8 to the flanks of
rabbits, n = 4, dose, 10.7-11.2 mg of toltrazuril/kg of BW; the animals
were fixed until 4 hours after application

Serum concentration of
toltrazuril sulphone
[μg/L] in animal No.:

| Time | 2564 | 2589 | 2548 | 2562 | Mean [μg/L] |
|---|---|---|---|---|---|
| Prior to | <LoQ | <LoQ | <LoQ | <LoQ | <LoQ |
| appl. | | | | | |
| 1 h | <LoQ | <LoQ | 30 | <LoQ | 35 |
| 2 h | <LoQ | <LoQ | 31 | <LoQ | 35 |
| 4 h | <LoQ | <LoQ | 31 | <LoQ | 34 |
| 5 h | <LoQ | <LoQ | 32 | <LoQ | 126 |
| 6 h | <LoQ | <LoQ | 31 | <LoQ | 263 |
| 8 h | <LoQ | <LoQ | 41 | <LoQ | 892 |
| 10 h | <LoQ | <LoQ | 64 | <LoQ | 1471 |
| 14 h | 30 | 26 | 155 | 25 | 2048 |
| 28 h | 251 | 223 | 554 | 198 | 3317 |
| 34 h | 491 | 391 | 747 | 245 | 3564 |
| 52 h | 1240 | 780 | 1349 | 709 | 3853 |

The data show that, after an active compound-containing formulation has been applied to the coats of rabbits, the substance is taken up orally as a result of the grooming behaviour; no percutaneous uptake was seen.

1. Pharmaceutical preparation for use in animals, which is applied to the coat or the skin of the animal and which the latter then takes up orally.

2. Pharmaceutical preparation according to claim 1, which is intended for use in cats.

3. Pharmaceutical preparation according to claim 1, which exhibits a liquid consistency.

4. Pharmaceutical preparation of claim 1, comprising flupirtine or its salts.

5. Pharmaceutical preparation of claim 1, comprising enrofloxacin or its salts.

6. Pharmaceutical preparation of claim 1, comprising pradofloxacin or its salts.

7. Pharmaceutical preparation of claim 1, comprising toltrazuril or its salts.

8. Pharmaceutical preparation of claim 1, comprising ponazuril or its salts.

9. (canceled)

10. Method for applying pharmaceutical active compounds in animals, in which a pharmaceutical preparation comprising the corresponding active compound is applied topically to the animal and the animal then takes up orally the pharmaceutical which has been thus applied.
